(19) United States
(12) Patent Application Publication
(10) Pub. No.: US 2017/0016031 A1
(43) Pub. Date: Jan. 19, 2017

(54) OPTIMIZED BIOGAS (BIOMETHANE) PRODUCTION FROM ANAEROBIC REACTORS

(71) Applicants: Enos Loy Stover, Stillwater, OK (US); Ted Ross Stover, Stillwater, OK (US)

(72) Inventors: Enos Loy Stover, Stillwater, OK (US); Ted Ross Stover, Stillwater, OK (US)

(73) Assignee: Enos Loy Sinver

(21) Appl. No.: 15/277,801

(22) Filed: Sep. 27, 2016

Related U.S. Application Data
(62) Division of application No. 13/044,197, filed on Mar. 9, 2011, now abandoned.
(60) Provisional application No. 61/312,088, filed on Mar. 9, 2010.

(51) Int. Cl. C12P 5/02; C12P 3/00

(52) U.S. Cl. CPC C12P 5/023; C12P 3/00

(57) ABSTRACT

An improved process and apparatus for producing biogas having a high methane content from a feed or substrate. Feed material is injected into a reactor having anaerobic microorganisms to form a bulk liquid in the reactor. The oxidation-reduction potential, pH, and temperature of the bulk liquid and the methane, carbon dioxide, hydrogen sulfide, and flow of the biogas is monitored. The amount of the feed material (substrate) fed to the reactor is adjusted in response to the monitoring parameters of the bulk liquid and biogas. A biomass recycle is provided to the reactor, thus increasing the reactor biomass retention time, or solids retention time within the reactor.

### CROSS-REFERENCE TO RELATED APPLICATIONS

The present application is a divisional application of U.S. Ser. No. 13/044,197, filed Mar. 9, 2011, which claims benefit under 35 U.S.C. §1.19(e) of U.S. Provisional Patent Application Ser. No. 61/312,088, filed Mar. 9, 2010, entitled “OPTIMIZED BIOGAS (BIOMETHANE) PRODUCTION FROM ANAEROBIC REACTORS”. The entire contents of each of the above-referenced applications are hereby expressly incorporated herein by reference.

### FIELD OF THE INVENTION

The present invention relates generally to biological anaerobic digestion processing of waste materials and various energy feedstocks (substrates), more particularly, but not by way of limitation, to anaerobic mesophilic and thermophilic treatment processes for wastes and energy feedstocks in various forms to produce biogas (biomethane).

### BACKGROUND OF THE INVENTION

Numerous anaerobic processes have been developed over the years for biological treatment of domestic and industrial waste residuals. Anaerobic processes occur in suspended growth reactors, fixed-film reactors, and combinations of both technologies referred to as hybrid reactors. Typically, the anaerobic reactor design utilized for biogas production applications is a once through continuous stirred tank reactor (CSTR). The methane in the biogas produced during anaerobic digestion is a valuable energy source. One advantage to anaerobic treatment processes is that they produce biogas which consists primarily of methane (CH₄), along with carbon dioxide (CO₂) and typically some hydrogen sulfide (H₂S).

Both soluble wastewater constituents and particulate or suspended solid matter (energy feedstocks or substrates) can be used as a food or fuel source for the anaerobic microorganisms to produce biomethane. Solid matter first has to be biochemically hydrolyzed to soluble constituents and transported across the microorganism cell wall before it can be used as a food or fuel source by the microorganisms. The food or fuel value of the waste material (energy feedstocks or substrates) is best measured as chemical oxygen demand (COD) or volatile solids (VS).

The COD can be measured or calculated. The calculated or theoretical COD represents the stoichiometric amount of oxygen which would be required to chemically oxidize all of the food, fuel, or organic matter in the waste material (feedstocks) to carbon dioxide and water. The COD value can be calculated when the composite empirical formula for the feedstocks or substrates being digested is known along with their relative concentrations. Otherwise, the COD can be measured by a standard COD test methodology used to oxidize all the organic matter to carbon dioxide and water, whereby the associated oxygen equivalents are measured. Likewise, the volatile solids content of a waste can be measured by a standard solids test methodology, whereby the solids are burned in a furnace for gravimetric determination of the amount of solids volatilized or lost. Either the COD, VS, or both can be effectively utilized to measure the amount of substrate, food source, fuel, or organic matter available in the feedstocks for utilization by the anaerobic bacteria for biomethane production, growth, energy production, heat production, and cell maintenance.

To this end, although processes of the existing art produce biogas, further improvements are desirable to produce the maximum biogas from a given feedstock(s) or substrate(s) at the highest biomethane content. It is to such a process that the present invention is directed to utilize the operational process control techniques/methodologies of the present invention to enhance and optimize the anaerobic conversion of feedstocks or substrates, individually or combined (co-digestion), to biogas (biomethane) with any digester design concepts. Further, an improved digester design (apparatus) is provided for the reactor by providing biomass recycle to the reactor, thus increasing the reactor biomass retention time, or solids retention time within the reactor.

### SUMMARY OF THE INVENTION

The present Invention is an anaerobic process for producing biogas. A feed material is injection into a reactor having anaerobic microorganisms to form a volume of bulk liquid in the reactor. Oxidation-reduction potential, pH, and temperature of the reactor bulk liquid is monitored to determine whether oxidation-reduction potential, pH, and temperature are each within a predetermined range. The amount of feed material fed to the reactor is adjusted in response to a determination that one of the oxidation-reduction potential, pH, and temperature of the reactor bulk liquid are outside the corresponding predetermined ranges. The oxidation-reduction potential, pH, or temperature of the reactor bulk liquid is adjusted to within the predetermined range. Biogas having methane is produced from the process.

The pH of the reactor bulk liquid is maintained within a range of from about 6.5 to about 8.5. The oxidation-reduction potential of the reactor bulk liquid is maintained between about −300 mV and about −400 mV. When the process in the reactor is performed mesophilic, the temperature is between about 80° F. and about 100° F. When the process in the reactor is performed thermophilic, the temperature is between about 125° F. and about 150° F. The rate of the reactor biogas production Is monitored to determine whether rate of production of the biogas is within a predetermined range. The relative rates of methane, carbon dioxide, and hydrogen sulfide production of the reactor biogas are monitored to determine whether methane, carbon dioxide, and hydrogen sulfide production are each within a predetermined range. The methane constitutes between about 60% and about 85% of the biogas. The carbon dioxide constitutes between about 15% and about 40% of the biogas. Treated effluent is removed from the reactor and at least a portion of the treated effluent is recycled to the reactor to increase biomass inventory within the reactor. The treated effluent is passed through a solids/liquid separation apparatus for further treatment of the effluent.

The present invention is an anaerobic process for producing biogas. An influent mixture is injected into a reactor having anaerobic microorganisms to form a volume of bulk liquid in the reactor, wherein the influent mixture comprises a feed material and recycled effluent. The oxidation-reduction potential, pH, and temperature of the reactor bulk liquid is monitored. At least a selected one of the rate of injection of the recycled effluent and the amount of the feed material fed to the reactor is adjusted in response to the oxidation-reduction potential, pH, and temperature of the reactor bulk liquid to maintain the temperature of the reactor bulk liquid within a predetermined range. Biogas having methane is produced.

The pH of the reactor bulk liquid is maintained within a range of from about 6.5 to about 8.5. The oxidation-reduction potential of the reactor bulk liquid is maintained between about −300 mV and about −400 mV. When the process in the reactor is performed mesophilic, the temperature is between about 80° F. and about 100° F. When the process in the reactor is performed thermophilic, the temperature is between about 125° F. and about 150° F.

The rate of the reactor biogas production Is monitored to determine whether rate of production of the biogas is within a predetermined range. The relative rates of methane, carbon dioxide, and hydrogen sulfide production of the reactor biogas are monitored to determine whether methane, carbon dioxide, and hydrogen sulfide production are each within a predetermined range. The methane constitutes between about 60% and about 85% of the biogas. The carbon dioxide constitutes between about 15% and about 40% of the biogas.

The present invention is an apparatus for producing biogas. The apparatus includes a reactor, a feed conduit, a biogas conduit, an effluent conduit, a temperature sensor, a pH sensor, and an oxidation reduction potential sensor. The reactor contains anaerobic microorganisms and bulk liquid. The feed conduit is operably coupled to the reactor which facilitates introduction of feed material into the reactor. The biogas conduit facilitates removal of biogas from the reactor. The effluent conduit facilitates removal of treated effluent from the reactor. The temperature sensor measures the temperature of the bulk liquid. The pH sensor measures the pH of the bulk liquid. The oxidation-reduction potential sensor measures the oxidation-reduction potential of the bulk liquid. Further, the apparatus includes a recycle effluent conduit for recycling a portion of the treated effluent back to the reactor and a flow meter which is operably coupled to the biogas conduit for measuring the level of flow of biogas from the reactor. A methane sensor is also provided and is operably coupled to the biogas conduit for measuring the amount of methane in the biogas. A carbon dioxide sensor is operably coupled to the biogas conduit for measuring the amount of carbon dioxide in the biogas. A hydrogen sulfide sensor is operably coupled to the biogas conduit for measuring the amount of hydrogen sulfide in the biogas. A plurality of Injection conduits facilitate injection of process chemicals selected from the group consisting of caustic, magnesium hydroxide, micronutrients, ferrous chloride, ferric chloride, macronutrients, and lime. A microprocessor is connected to the oxidation-reduction potential, pH, and temperature sensors. The microprocessor automatically adjusts the rate of feed material into the reactor in response to predetermined ranges of parameters selected from the group consisting of temperature, pH, or ORP.

The microprocessor automatically adjusts the rate of feed material into the reactor in response to predetermined ranges of parameters selected from the group consisting of biogas flow rate, CH₄, or CO₂. The microprocessor automatically adjusts the rate of feed material into the reactor in response to predetermined ranges of parameters selected from the group consisting of biogas production rate, methane production rate, COD, VS, VFAs, or alkalinity.

The present Invention provides an improved process and apparatus for enhanced and optimized anaerobic mesophilic and thermophilic biological digestion of wastes or energy feedstocks (e.g., biomass, materials containing lipids (fats), proteins, carbohydrates, such as starch, cellulose, hemicellulose, etc., lignocellulose, materials having biodegradable solids, etc., in various forms, (e.g., soluble, slurry, particulate/solid or combination forms). The improved anaerobic process utilizes kinetic modeling and specific parameter monitoring to optimize and control the digestion process, including suspended growth, fixed-film, and hybrid anaerobic reactor technologies operating in either the mesophilic or thermophilic mode.

In particularly preferred embodiments, parameters such as pH, oxidation reduction potential (ORP), temperature, chemical oxygen demand (COD), total solids (TS), total volatile solids (VS), total suspended solids (TSS), volatile suspended solids (VSS), VFAs, total Kjeldahl nitrogen (TKN), NH₃—N, PO₄—P, alkalinity and sulfides in the reactor bulk liquid, as well as methane, CO₂, and H₂S in the biogas are monitored to provide further process information for optimization of the anaerobic digestion processes. Developed biological kinetic relationships and specific formulations of biological growth micronutrients are utilized for further enhancing and optimizing the anaerobic digestion processes.

Similarly, the apparatus of the present invention Includes a means for injecting an influent mixture into a reactor having anaerobic microorganisms to form a volume of bulk liquid in the reactor, wherein the influent mixture includes energy feedstocks, means for on-line monitoring the pH, oxidation-reduction potential, and temperature of the reactor bulk liquid, means for on-line monitoring the biogas flow rate and biogas methane, CO₂ and H₂S content, means for monitoring the key analytical parameters such as VFAs, alkalinity, COD, VS, etc., means for monitoring the biokinetic constants for biogas production, methane production, and treatment performance, and means for adjusting at least a selected one of the rate of the various feedstocks based on an algorithm controlled by a programmable logic controller (PLC) to optimize the reactor bulk liquid for biogas production, treatment performance, and pH and alkalinity control as a function(s) of all the previously discussed parameters. Biomass recycle to the digester reactor can also be provided for enhanced performance and biomethane production optimization, wherein the digester effluent passes through a solids/liquid separation device (such as a centrifuge, dissolved air or gas flotation system, gravity belt thickener, ultrafilter membrane, etc.). The cleaned effluent is removed of the majority of the biomass solids and is of good quality for recycle/reuse such as direct irrigation, and the majority of the separated biomass solids are recycled back to the digester reactor to increase the biomass inventory within the reactor. A portion of the biomass solids are wasted from the system as concentrated solids with high fertilizer nutrient value. The biomass solids returned to the reactor provides the following significant advantages to the digester operation/performance:
1. Biomass inventory management and control;
2. F/M process control through biomass recycle;
3. Improved digestion process stability and performance enhancement;
4. Alkalinity recycle and pH control;
5. Enhanced and optimized biomethane production; and
6. Increased nutrient (nitrogen and phosphorus) value of the residual biomass solids.

These and various other features, as well as advantages which characterize the present invention, will be apparent from a reading of the following description.

### BRIEF DESCRIPTION OF THE SEVERAL VIEWS OF THE DRAWINGS

FIG. 1 is a flowchart for an anaerobic control process for producing biogas executed in accordance with one embodiment of the present invention.

FIG. 2 is a schematic diagram of one embodiment of a reactor constructed in accordance with the present invention utilized for practicing the process of the present invention.

FIG. 3 is a flow chart for an anaerobic control process.

### DETAILED DESCRIPTION OF THE INVENTION

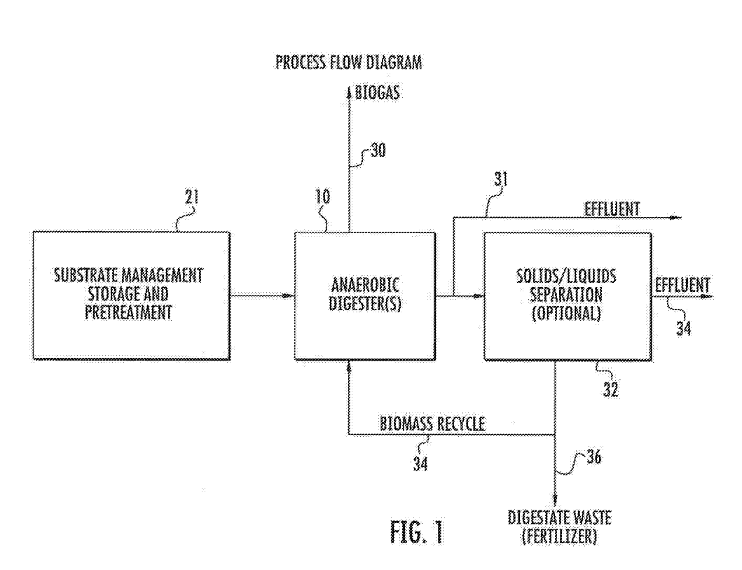

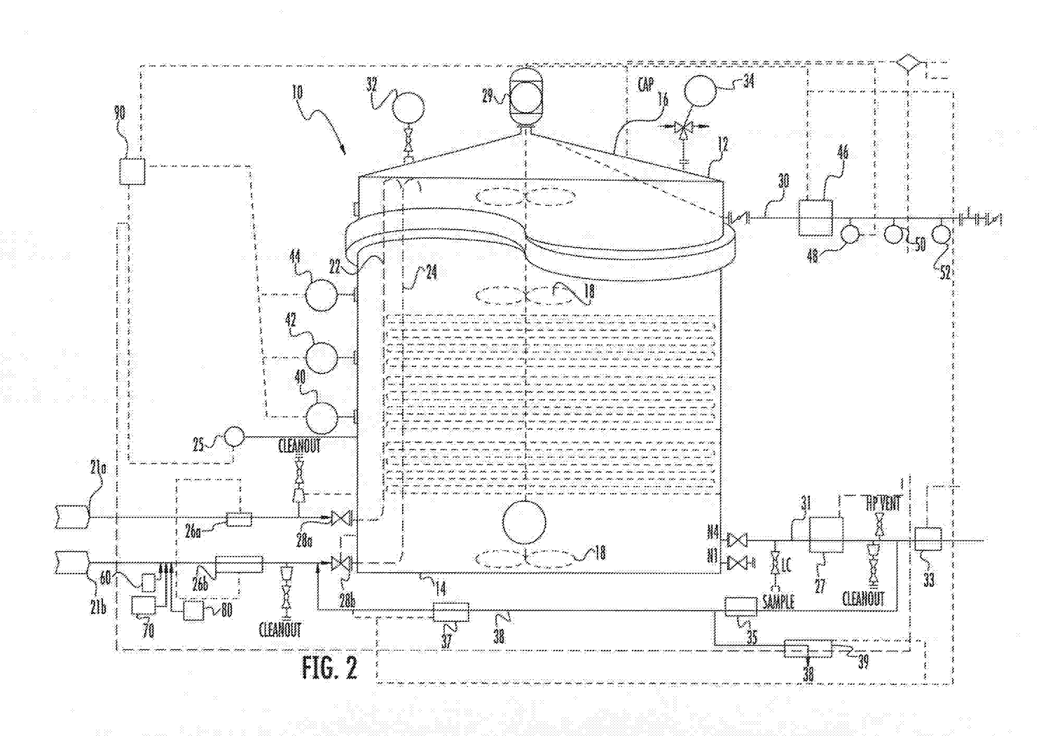

Referring now to the drawings, and more particularly to FIGS. 1 and 2, the present invention provides an improved process and apparatus for anaerobic biological digestion of a feed material. Examples of the feed material include wastes or energy feedstocks (e.g., biomass, materials containing lipids (fats), proteins, carbohydrates, such as starch, cellulose, hemicellulose, etc., lignocellulose, materials having biodegradable solids, etc., in various forms (e.g., soluble, slurry, particulate/solid or combination forms). It should be understood by one of ordinary skill in the art that the feed material may be any material capable of undergoing anaerobic digestion for producing biogas in accordance with the present invention as described herein.

Shown therein is a reactor 10 constructed in accordance with the present Invention for anaerobic digestion of the feed material for producing biogas. The reactor 10, in one embodiment of the present invention as described herein is a CSTR. However, it should be understood by one of ordinary skill in the art that any reactor may be utilized for anaerobic digestion so long as the reactor functions in accordance with the present invention. Further, it should be understood by one of ordinary skill in the art that although a single reactor 10 is shown as described herein, a plurality of reactors may be utilized so long as the plurality of reactors functions in accordance with the present invention as described herein.

The reactor 10 includes a vessel 12 having a bottom 14, a top 16, and at least one agitator 18 for mixing the feed material. The reactor 10 may further be provided with a heating apparatus 20, such as heat coils, for heating the biomass and feed material in the reactor 10. The heating apparatus 20 is utilized in the event the heating requirements (mesophilic or thermophilic) are not satisfied by a fuel value content or the temperature of the feed material to be treated. Such a supplemental heating system allows the reactor bulk liquid temperature to be maintained within the desired temperature range.

A feed source(s) 21a and 21b designate the feed material to be treated by the process, which may be any soluble, slurry, or solid waste, or various combinations thereof, having organic and/or other constituents particularly suitable for anaerobic treatment as further described herein.

The feed material is pumped to the anaerobic reactor 10 by a feed pumping system by conduits 22 and 24 for ultimate mixing with the reactor contents (or “bulk liquid”) of reactor 10. To regulate the flow of feed material to the reactor 10, a flow meter 26 (26a and 26b) and control valve 28 (28a and 28b) are provided in the conduits 22 and 24. Alternatively, the flow of influent feed material can be regulated by using a variable speed pump (not shown). A liquid level sensor 25 monitors the level of the bulk liquid in the vessel 12.

Upon entering the reactor 10, the feed material is mixed by the at least one agitator 18 with the anaerobic microorganisms to digest the feed material. A motor 29 controls the speed and direction of the at least one agitator 18. Biogas is generated by the anaerobic process and is withdrawn from the reactor 10 via a biogas line 30. A pressure indicator 32 is provided for monitoring the pressure in the reactor 10. Also, pressure relief valve 34 is provided for relief of gas from the reactor 10, in the event the pressure in the reactor 10 exceeds a predetermined pressure amount.

Effluent stream 31 of the reactor 10 is recycled to the reactor 10, used as a fertilizer, or to an effluent transfer system (not shown). Flow meters 27 and 33 are provided along the effluent stream 31 to regulate the flow of the effluent.

The recycle portion of the effluent 31 passes through a solids/liquid separation apparatus 35 (such as a centrifuge, dissolved air or gas flotation system, gravity belt thickener, ultrafilter membrane, etc.). The majority of the biomass solids are removed from the effluent 31 and is of good quality for recycle/reuse such as direct Irrigation. The majority of the separated biomass solids are recycled back to the reactor 10 by stream 36 to increase the biomass inventory within the reactor 10. A flow meter 37 and control valve 41 are provided to monitor and regulate the flow of the recycle stream 36. A portion of the biomass solids are wasted from the process as a concentrated fertilizer by stream 38. A flow meter 39 is provided to monitor and regulate the flow of the fertilizer stream 38.

The improved anaerobic digestion process utilizes specific parameter monitoring and biokinetic relationships to optimize and control the treatment process for enhanced/optimized biogas (methane) production. Preferably, parameters are monitored in both the reactor bulk liquid and the biogas produced. Biological kinetic relationships developed as described herein, and specific formulations of biological growth micronutrients are utilized for further enhancing and optimizing the anaerobic digestion processes.

In accordance with the present invention, optimization of the anaerobic digestion process is achieved by on-line monitoring and controlling the temperature, pH and ORP of the reactor bulk liquid to maintain these critical parameters within appropriate ranges. Thus, the reactor 10 is provided with a temperature sensor 40, a pH sensor 42 and an ORP sensor 44 to monitor these critical parameters of the bulk liquid.

Secondary parameters are also monitored to give further information as to the state of the reaction process taking place in the reactor 10. A biogas flow meter 46, a methane sensor 48, a carbon dioxide sensor 50, and a hydrogen sulfide sensor 52 are provided in the biogas line 30 for monitoring the biogas flow rate and levels of each of these constituents in the biogas. While preferred ranges for each of these parameters may vary, in many feedstock applications methane in the biogas will preferably be in the range of about 60 to about 85 percent and carbon dioxide will preferably be about 15 to about 40 percent. The hydrogen sulfide content of the biogas will vary as a function of the amount of sulfur in the various feedstocks employed for biogas production. Furthermore, volatile fatty acids (VFAs) and sulfide concentrations in the reactor bulk liquid are typically monitored using wet chemistry analysis of samples taken from the reactor bulk liquid. In many anaerobic digestion plants, the preferred range for VFAs will be about 500 to 3,500 mg/L, and the preferred range for sulfides will be about 1.0 mg/L. Each of these parameters provides further information as to the state of the anaerobic treatment process.

The methane sensor 48 determines the concentration of methane in the biogas and, along with the biogas flow rate, provides an indication of the energy production (biomethane) as a function of the treatment performance. The carbon dioxide sensor 50 determines the level of carbon dioxide in the biogas and can be used to determine the amount of carbon dioxide generated as a function of the treatment performance and the operating characteristics of the bulk liquid in the anaerobic reactor 10.

Hydrogen sulfide (H₂S) is partially soluble and insoluble, and as the H₂S is produced above its solubility level, it diffuses out of solution and into the biogas. This is a normal aspect of anaerobic systems and the amount of sulfides in the bulk liquid and H₂S in the biogas can be monitored and controlled to achieve maximum treatment performance. The sulfides level in the anaerobic reactor bulk liquid is typically determined using wet chemistry techniques, while the H₂S level in the biogas is determined using the H₂S sensor 52.

Under anaerobic conditions and the low (negative) ORP levels associated with the anaerobic processes of the present invention, fermentation reactions will exist with VFA formation. VFAs produced from fermentation reactions must be monitored, managed, and controlled for optimum biogas (biomethane) production. VFA formation, if allowed to accumulate to high levels in the reactor 10, can cause biological feedback inhibition and reduced treatment performance and biogas production. In accordance with the present process, VFAs are typically conveniently monitored using wet chemistry techniques. Alkalinity is also typically monitored with wet chemistry techniques, and both VFAs and alkalinity are used in the process control algorithms with PLC process controls.

Thus, monitoring methane, carbon dioxide, and hydrogen sulfide in the biogas, as well as VFAs, alkalinity, and sulfides in the reactor bulk liquid in accordance with the process of the present invention, provides further information as to the conditions in the anaerobic reactor 10. Such information, in combination with the bulk liquid temperature, pH, and ORP are used to ensure an optimized environment in the anaerobic digestion reactors for maximum biogas and biomethane production.

The higher the methane content in the biogas, the higher the Btu (energy) value of the biogas; and the lower the H₂S content of the biogas, the cleaner the biogas minimizing needs for H₂S cleaning, scrubbing, etc., for emissions controls. Therefore, optimized management of the overall biogas production process can have significant impacts on biogas quality and associated value. Tables 1 and 2 provide an overview of various operational control parameters that are utilized by the current invention for optimized/enhanced biogas quality (maximum methane and Btu content, while minimizing emissions). For example, increasing the pressure in the anaerobic reactor increases the CO₂ and H₂S solubility in the bulk liquid, thus decreasing the CO₂ and H₂S content in the biogas and increasing the methane content. However, increasing the temperature decreases the solubility of CO₂ and H₂S, with the resultant opposite and negative impacts on biogas quality. Increasing the pH of the bulk liquid Increases the solubility of both CO₂ and H₂S with the resultant effect of decreasing the CO₂ and H₂S content of the biogas, resulting in higher methane content. Increasing the divalent cations, calcium and magnesium, has no effect on the solubility of CO₂ and H₂S in the bulk liquid, but these cations can decrease the CO₂ content of the biogas due to precipitation of calcium carbonate and formation of magnesium carbonate, thus reducing the amount of free available CO₂ in the bulk liquid, and thus reducing the CO₂ content of the biogas. Increasing the calcium has no impact on the H₂S content of the biogas, while increased magnesium can decrease the H₂S content of the biogas. Adding iron salts to the reactor bulk liquid does not affect the solubility of CO₂ or H₂S, but can significantly reduce the H₂S content of the biogas by precipitating out soluble sulfides in the bulk liquid as iron sulfide (ferrous sulfide).

TABLE 1
CO₂ in Biogas

| | | Effect | |
|---|---|---|---|
| Parameter | Change | CO₂ Solubility | % CO₂ in Biogas |
| Pressure | + | Increase | Decrease |
| Temperature | + | Decrease | Increase |
| pH | + | Increase | Decrease |
| Calcium | + | No Effect | Decrease |
| Magnesium | + | No Effect | Decrease |
| Preacidification | + | No Effect | Decrease |
| (Sodium and Potassium) | | | |
| Ammonia | + | No Effect | Decrease |
| Iron Salts | + | No Effect | No Effect |

TABLE 2
H₂S in Biogas

Effect

| Parameter | Change | H₂S Sol. | % H₂S |
|---|---|---|---|
| Pressure | + | Increase | Decrease |
| Temperature | + | Decrease | Increase |
| pH | + | Increase | Decrease |
| Calcium | + | No Effect | No Effect |
| Magnesium | + | No Effect | Decrease |
| Preacidification | + | No Effect | No Effect |
| (Sodium and Potassium) | | | |
| Ammonia | + | No Effect | No Effect |
| Iron Salts | + | No Effect | Decrease |

Generated alkalinity in the reactor bulk liquid can also have significant impacts on decreasing the CO₂ content of the biogas, as well as maintaining pH and alkalinity control in the reactor bulk liquid (subject matter of a separate patent application). Ammonia-nitrogen reacts with CO₂ to produce ammonium bicarbonate alkalinity in the bulk liquid which generates alkalinity assisting with pH control while at the same time reducing the amount of CO₂ emitted in the biogas. Therefore, the formation of ammonium bicarbonate reduces the CO₂ content of the biogas while having no effect on the H₂S content of the biogas. The monovalent cations, sodium and potassium, under proper pretreatment conditions (subject matter of a separate patent application) producing VFA salts of sodium and potassium prior to entering the reactor, contribute to alkalinity production as sodium and potassium bicarbonate to help with pH and alkalinity control in the bulk liquid. These reactions have no effect on the bulk liquid solubility of CO₂ and H₂S; however, the generated alkalinity consumes CO₂, thus reducing the amount of CO₂ emitted in the biogas.

Utilizing the physical and chemical factors described in Tables 1 and 2, operational process control in the anaerobic reactor 10 and appropriate pretreatment steps are utilized to enhance and optimize the methane content of the biogas produced in the anaerobic reactor. Various combinations of process control techniques and methodologies are utilized to maximize the methane content of the biogas by reducing both the CO₂ and H₂S content in the biogas produced.

Table 3 summarizes, typical preferred ranges for the process control parameters.

TABLE 3

| Parameter | Ranges |
|---|---|
| Temperature, T | Mesophilic - about 80° F. to about 100° F. |
| | Thermophilic - about 125° F. to about 150° F. |
| Oxidation Reduction | about −300 mV to about −400 mV |
| Potential, ORP | |
| pH | About 6.5 to about 8.5, optimum will vary with |
| | type of substrate and acclimation of |
| | methanogens |
| Biogas, methane | between about 60% and about 85% |
| Biogas, carbon dioxide | between about 15% and about 40% |
| Biogas, hydrogen | Sulfide content varies as a function of how |
| sulfide | much sulfur is in the feed |
| Actual methane | About 6.3 cubic feet per pound |
| produced | COD removed at 95° F. |
| Actual biogas produced | About 7.4 to about 10.5 cubic feet per pound of |
| (biogas volume or | COD removed at 95° F. |
| flow rate) | |

A ferrous chloride (FeCl₃) and/or ferric chloride (FeCl₂) source 60 can be added to the reactor feed or bulk liquid to provide these chemicals as micronutrients and for sulfide complexation, if needed. Either FeCl₃ or FeCl₂ can be used for sulfide control by complexing or precipitating sulfides as they are formed in the reactor bulk liquid.

A macronutrients (nitrogen and phosphorus) source 70 is provided, if needed, for nutrient deficient feedstocks and a micronutrients (trace metals) source 80 (micronutrient cocktail) is provided because such nutrients are critical to successful performance of anaerobic digestion systems, especially when treating nutrient deficient wastes. The levels of the macronutrients nitrogen and phosphorus are normally inadequate in high strength carbohydrate feedstocks. Aqueous ammonia and phosphoric acid can be used to supply nitrogen and phosphorus, as well as various forms of fertilizers. The micronutrient source 80 provides the following primary chemicals (for example, along with additional heavy metals and organic growth factors, various combinations as may be needed) necessary for biological growth requirements:
Ferric chloride/ferrous chloride
Calcium chloride
Ammonium molybdate
Nickel chloride
Copper sulfate
Cobalt chloride
Zinc sulfate

The trace metals are critical in controlling the rate of enzyme reactions which set the rate of biological activity. Trace metals also serve as regulators of osmotic pressure and to transfer electrons in oxidation-reduction reactions such as the storage of energy, i.e., the conversion of ADP to ATP. The major trace elements required by anaerobic bacteria include iron, magnesium, calcium, copper, zinc, nickel, cobalt, molybdenum, selenium and tungsten. Any of these micronutrients can be added to the anaerobic reactor in low concentrations, as necessary, to stimulate the anaerobic digestion process and enhance and optimize biogas production.

In addition to maintaining optimum temperature and pH, along with eliminating toxicity, it is important to provide all the proper micronutrients and macronutrients for optimum anaerobic microorganism metabolism. The addition of a plurality of biochemical enhancement chemicals (salts of iron, magnesium, calcium, sodium, copper, zinc, nickel, cobalt, molybdenum, selenium, tungsten, vanadium, manganese, and potassium, all or partially as needed) or cocktail of chemicals, provides enhancement and optimization of biochemical reaction rates that offers significant advantages in terms of overall treatment performance, process optimization, biogas production rate and quality, and capabilities to treat complex, inhibitory, and difficult to treat feedstocks that would not otherwise be treatable anaerobically. Enhancement and optimization of biochemical enzyme reaction rates which control the rate of biological activity are critical to taking full advantage of anaerobic reactor design and operations. Trace metals also serve as regulators of osmotic pressure and to transfer electrons in oxidation-reduction reactions for the production and storage of energy. Each feedstock to be subjected to anaerobic treatment can be chemically analyzed and a specific biochemical enhancement cocktail formulated based on the deficiencies found for that particular feedstock (substrate).

Trace metals are essential for proper anaerobic metabolic reactions. Enzyme activity and electron transport cannot occur without a number of heavy metals. Potassium and magnesium are required in relatively large quantities when compared with the essential trace metals of cobalt and molybdenum. Potassium is important as an enzyme activator and for maintenance of osmotic pressure and regulation of pH. Magnesium is the most abundant Intracellular cation, is an enzyme activator, and binds enzymes to substrates. Manganese is an enzyme activator and cofactor for some enzymes and can sometimes replace magnesium. Calcium is an Important intracellular cation, is a cofactor for some enzymes, and sometimes replaces magnesium. Iron is required in many redox reactions catalyzed by haem proteins and is a cofactor for many enzymes. Iron is virtually important to all living organisms. Cobalt is a constituent of vitamin B-12 and is essential for microbial growth. Molybdenum, copper, nickel, and zinc are important inorganic constituents of metalloenzymes required for metabolic activity. All of these metals must be present in adequate quantities in the feedstock or added to the feedstock if an anaerobic biological treatment system is to have a balanced ecology. Limited quantities, or the absence of one or more of these essential elements, may restrict growth, reduce the efficiency of treatment, and/or allow the predominance of undesirable and nuisance microorganisms.

The various monitor and control elements of the anaerobic reactor are regulated automatically by means of a PLC 90, which includes a computer linked to the various monitoring and control elements. Although a PLC is utilized in one embodiment of the present invention, by way of example, it should be understood by one of ordinary skill in the art that any type of microprocessor controller may be used in accordance with the present invention.

Various parameter setpoints are initially established by the operator. The parameter setpoints can Include a desired temperature range within which the process operates and desired pH and ORP operating ranges for the process.

The parameter setpoints are provided to a microprocessor, such as the PLC 90, which proceeds to monitor the operation of the anaerobic process. More particularly, the temperature, pH, ORP, methane, carbon dioxide, and biogas flow are periodically measured and checked to determine whether these measured parameters are within the selected operating ranges. When the measured parameters remain within the selected ranges, no adjustments are made to the control elements. However, when the measured parameters fall outside the selected operating ranges, operational process control changes are required.

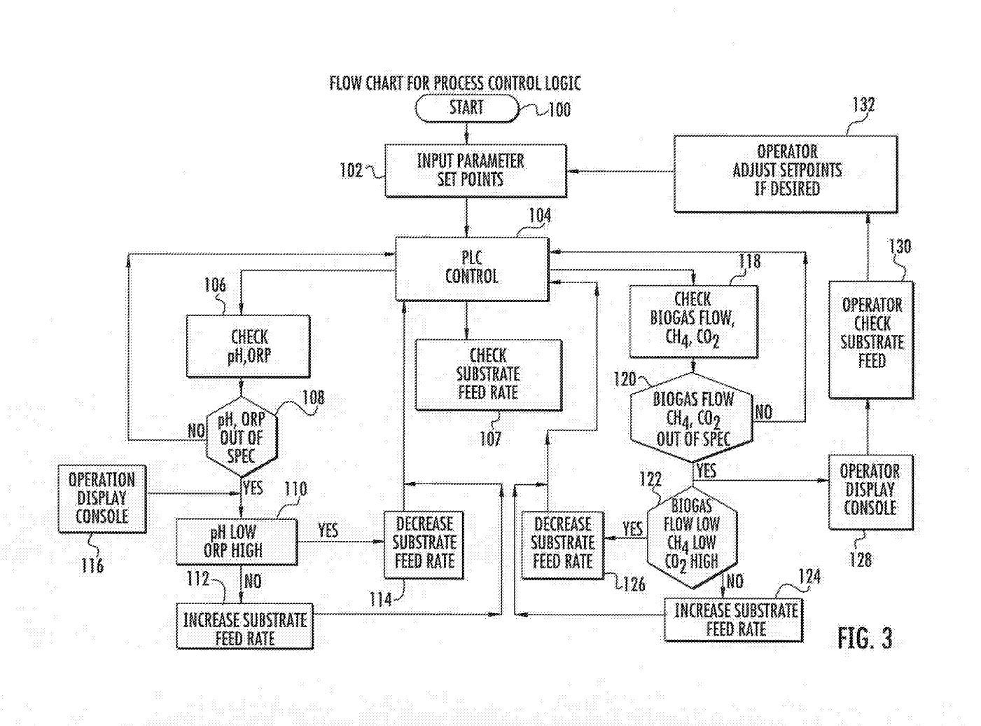

An example of one embodiment of such an arrangement is shown in FIG. 3. More particularly, FIG. 3 provides a flow chart for an anaerobic control process 100 in accordance with a preferred embodiment of the present invention. Each of the steps of the process will be discussed in turn.

Beginning at step 102, various parameter setpoints are initially established by the operator. As discussed above, such parameter setpoints can Include a desired temperature range within which the process operates and a desired ORP, pH, biogas flow, CH₄, and CO₂ range for the process. It will be understood that the desired ORP range will typically be expressed in negative millivolts, and will have a first threshold (such as −300 millivolts) and a second threshold (such as −400 millivolts), with the first threshold having a greater absolute value than the second threshold. The pH range is between about 6.5 and about 8.5. Methane in the biogas is between about 60% and about 85%. Carbon dioxide in the biogas is in the range of about 15% to about 40%. The biogas flow is maintained between about 7.4 and about 10.5 cubic feet per pound of COD removed at about 95° F.

At step 104, the parameter setpoints are provided to a microprocessor, such as the PLC 90, which proceeds to monitor the operation of the anaerobic process. More particularly, as indicated at step 106, the pH and/or ORP are periodically measured and checked to determine whether the measured pH and ORP are within the selected pH and ORP ranges. When the measured pH and ORP remain within the selected ranges, as shown by decision step 108, no adjustments are made to the control elements. Additionally, as indicated at step 107, the rate that the feed material enters the reactor is also monitored.

However, when the measured pH and ORP fall outside the selected pH and ORP ranges, the flow continues from decision step 108 to decision step 110, which determines whether the out of spec pH and/or ORP are outside the established ranges. If so, the flow continues to step 112 where the microprocessor operates to control the flow meters 26a and 26b and control valves 28a and 28b to increase the rate of feed material from the feed sources 21a and 21b by the setpoint value increment selected at step 102. On the other hand, if the out of spec pH and/or ORP change to where the pH is low and the ORP is high, the flow continues to step 114 where the microprocessor operates to decrease the rate of feed material from the feed sources 21a and 21b by the setpoint value increment selected at step 102. Preferably, the microprocessor initiates an internal timer upon detection of an out of spec pH and/or ORP and does not proceed to adjust the rate of feed material into the reactor 10 until expiration of the timer. This prevents undesired adjustments to spurious pH and/or ORP readings.

Additionally, from step 104, simultaneously, the PLC 90 monitors other parameters of the process. As indicated at step 118, the biogas flow, CH₄, and CO₂ are periodically measured and checked to determine whether the measured biogas flow, CH₄, and CO₂ are within the selected biogas flow, CH₄, and CO₂ ranges. When the measured biogas flow, CH₄, and CO₂ remain within the selected ranges, as shown by decision step 120, no adjustments are made to control elements.

However, when the measured biogas flow, CH₄, and/or CO₂ fail outside the selected biogas flow, CH₄, and/or CO₂ ranges, the flow continues from decision step 120 to decision step 122, which determines whether the out of spec biogas flow, CH₄, and/or CO₂ are outside the established ranges. If so, the flow continues to step 124 where the microprocessor operates to increase the rate of feed material into the reactor 10 from the feed sources 21a and 21b by the setpoint value increment selected at step 102. On the other hand, if the out of spec biogas flow, CH₄, and/or CO₂ are within the established ranges (biogas flow—low; CH₄—low; and CO₂—high) the flow continues to step 126 where the microprocessor operates to decrease the rate of feed material into the reactor 10 by the setpoint value increments selected at step 102. Preferably the microprocessor initiates an Internal timer upon detection of an out of spec biogas flow, CH₄, and/or CO₂ and does not proceed to adjust the rate of feed material into the reactor until expiration of the timer. This prevents undesired adjustments to spurious biogas flow, CH₄, and/or CO₂ readings.

Continuing with the flow of FIG. 3, at such time that the measured parameters are determined to be out of spec, the process also continues from the decision steps 108 to step 116 and from 120 to 128, where an indication is preferably made on an operator display console to inform the operator that the measured parameters are out of spec. This allows the operator to perform a manual check of the control elements, including the feed material/substrate flow meters 26a and 26b, and adjust Its flow rate by adjusting the flow control valve(s) 28a and 28b or adjusting the flow rate speed with a variable frequency drive, as shown at step 130, and to make any changes to the parameter setpoints at step 132.

As noted above, the most effective indicators of anaerobic reactor performance are bulk liquid temperature, pH, ORP, VFAs, alkalinity, COD, and sulfides, along with biogas methane, carbon dioxide, and H₂S content. Additionally, though, TS, VS, TSS, VSS, NH₃-N, P0₄-P and micronutrients can be monitored in the bulk liquid to provide still further process information. Each of these parameters should be kept within desired operating ranges which are plant specific in nature. An understanding of the interrelationships and interdependence of all these parameters, along with proper monitoring and control is required for successful start-up and operation of anaerobic reactors.

One aspect of anaerobic digestion of protein containing feedstocks is the release of organically bound nitrogen as NH₃-N. Therefore proteinaceous feedstocks generate excess nitrogen in the ammonia form which reacts with the excess CO₂ in the reactor bulk liquid to reduce the amount of free bulk liquid CO₂ and headspace CO₂ partial pressure by producing ammonium bicarbonate alkalinity (NH₄HCO₃). A significant portion of the CO₂ that is produced from the biological activity reacts with the ammonia and remains in the aqueous phase (bulk liquid). For each mg/L of NH₃-N formed, 5.6 mg/L of NH₄HCO₃ alkalinity is formed, which is equivalent to 3.6 mg/L of calcium carbonate (CaCO₃) alkalinity. The ammonium bicarbonate alkalinity causes the reactor bulk liquid pH to increase; with highly proteinaceous wastes the pH can increase into the 8.0 plus pH range.

High concentrations of NH₃-N, PO₄—P, and micronutrients released into the anaerobic reactor bulk liquid when treating various feedstocks can also be captured for fertilizer value.

One element for designing and operating anaerobic digestion systems has been found to be matching of the number of microorganisms in the system to the organic substrate (COD and/or VS) loading rate to the system, or controlling the food to microorganism (F/M) ratio or specific substrate application rate. Accurate prediction and modeling of both treatment performance and methane production has been accomplished when substrate utilization and methane production were expressed as functions of the specific mass substrate loading ratio (F/M) by monomolecular kinetics for both suspended growth and fixed-film systems. Extensive evaluation of anaerobic reactors over the past few years has shown that these systems comply with the same types of biokinetic relationships developed for description of aerobic suspended growth and fixed-film reactors. In the suspended growth systems the substrate loading rate is expressed as pounds of substrate applied per day, per pound of mixed liquor volatile suspended solids in the reactor. In the fixed-film systems the substrate loading rate is expressed as pounds of substrate applied per day, per 1000 square feet of reactor media surface area.

The number of microorganisms or biomass inventory within the reactor 10 can be increased, managed, and controlled by managing the biomass recycle and wasting rates. This is accomplished by a solids/liquid separation treatment step of the reactor effluent using any number of solids/liquid separation devices. The majority of the separated biomass is recycled back to the reactor in order to increase the biomass concentration, biomass inventory, and biomass solids retention time within the digester itself. By returning biomass to the reactor 10, the biomass retention time can be engineered to be greater than the hydraulic retention time of the digester reactor. This process then provides the mechanism for controlling the F/M ratio, by controlling the M or biomass within the system, which is the inherent concept and mechanism discussed in the following sections for optimized operation/performance and enhanced biogas production within the anaerobic reactor 10. By increasing the biomass inventory within the anaerobic reactor 10, the mass of substrate applied per day can be increased while still maintaining the same specific substrate application rate (F/M ratio), and thus increasing the amount of biogas (biomethane) that can be produced in the same reactor volume at the same treatment performance.

When considering an anaerobic reactor volume, either suspended growth or fixed-film, a mass balance of substrate into and out of that reactor volume can be made as follows:

mass of substrate=mass of substrate+mass of substrate into the reactor out of the reactor consumed biologically

In the case of the suspended growth system, the reactor volume is expressed in million gallons with the resultant mass balance equation:

FSᵢ=FSₑ+(dS/dt)_{G}V (1)

where
F=flow rate, million gallons per day (MGD)
Sᵢ=influent substrate concentration, mg/L
Sₑ=effluent substrate concentration, mg/L
V=reactor volume in million gallons
(dS/dt)_{G}=specific substrate utilization rate, lb/lb day
   Mathematical description of this substrate utilization rate as a function of the substrate loading rate or food to microorganism ratio (F/M) based on monomolecular kinetics follows:

$${( d S \text{/} d t )}_{G} = U = \frac{{U}_{max} \times ( {FS}_{i} \text{/} XV )}{{K}_{B} + ( {FS}_{i} \text{/} XV )} \qquad ( 2 )$$

where
FSᵢ/XV=F/M=food to microorganism ratio, lb/lb day
X=reactor mixed liquor volatile suspended solids, mg/L
U=specific substrate utilization rate, lb/lb day
Uₘₐₓ=maximum specific substrate utilization rate, lb/lb day
K_{B}=proportionality constant or substrate loading at which the rate of substrate utilization is one-half the maximum rate, lb/lb day

Substitution of equation 2 into equation 1 and solving for the reactor volume, V, or the effluent quality, Sₑ, provides the design equation or the effluent quality predictive equation to be used for operations, respectively.

The biokinetic constants, Uₘₐₓ and K_{B}, are determined experimentally. The specific substrate utilization rate can be plotted as a function of the F/M ratio in terms of COD for an anaerobic suspended growth system. The reciprocal of U is plotted as a function of the reciprocal of the F/M ratio in order to linearize this monomolecular kinetic relationship. In this linearized form Uₘₐₓ is the reciprocal of the y-axis intercept, and the slope of the line is equal to K_{B}/Uₘₐₓ.

In the case of a fixed-film anaerobic reactor, the reactor volume is expressed in terms of a thousand square feet of surface area. Different manufacturers' media have different specific surface areas (ft²/ft³), and in order to equitably compare different media, the conversion to media surface area in terms of a thousand square feet in a particular anaerobic reactor volume is mandatory. Based on this approach, a mass balance of substrate into and out of the fixed-film reactor volume can be made, as follows:

FSᵢ=FSₑ+(dS/dtA)_{G}A (3)

where
A=surface area of reactor volume, 1,000 ft²
(dS/dtA)_{G}=specific substrate utilization rate, lbs/day/1,000 ft²

Mathematical description of this substrate utilization rate as a function of the applied substrate loading rate based on monomolecular kinetics follows:

$${( d S \text{/} d tA )}_{G} = \frac{{U}_{max} ( FSi \text{/} A )}{{K}_{B} + ( FSi \text{/} A )} \qquad ( 4 )$$

where
FSi/A=applied substrate loading rate, lbs/day/1,000 ft²
Uₘₐₓ=maximum specific substrate utilization rate, lbs/day/1,000 ft²
K_{B}=proportionality constant, lbs/day/1,000 ft²

Substitution of equation 4 into equation 3 and solving for the reactor surface area, A, or the effluent quality, Se, provides the design equation or the effluent quality predictive equation to be used for operation, respectively.

The reciprocal of the specific substrate utilization rate is plotted as a function of the reciprocal of the specific substrate loading rate for determination of the biokinetic constants, Uₘₐₓ and K_{B}, as previously described for the anaerobic suspended growth systems.

Total biogas and methane production data from anaerobic digestion systems has indicated that both were functions of the specific substrate application rate, and therefore, they should respond in a similar manner as the substrate utilization kinetics. The number of microorganisms or biomass inventory within the digester apparatus can be increased, managed, and controlled by managing the biomass recycle and wasting rates. This is accomplished by a solids/liquid separation treatment step of the digester effluent using any number of solids/liquid separation devices. The majority of the separated biomass is recycled back to the digester reactor in order to increase the biomass concentration, biomass inventory, and biomass solids retention time within the digester itself. By returning biomass to the digester, the biomass retention time can be engineered to be greater than the hydraulic retention time of the digester reactor. This process then provides the mechanism for controlling the F/M ratio, by controlling the M or biomass within the system, which is the inherent concept and mechanism discussed in this section for optimized operation/performance and enhanced biogas production within the anaerobic digestion reactor itself. By increasing the biomass inventory within the anaerobic digester reactor, the mass of substrate applied per day can be increased while still maintaining the same specific substrate application rate (F/M ratio), and thus increasing the amount of biogas (biomethane) that can be produced in the same digester reactor volume.

Mathematical description of the biogas and methane production rates can be modeled as the substrate loading rate changes by using monomolecular kinetics in suspended growth systems. Total specific biogas production rate expressed as a function of the mass substrate loading rate for suspended growth systems follows:

$$G = \frac{{G}_{max} \times ( {FS}_{i} \text{/} XV )}{{G}_{B} + ( {FS}_{i} \text{/} xv )} \qquad ( 5 )$$

where
G=specific biogas production rate, ft³/day/lb substrate applied
Gₘₐₓ=maximum specific biogas production rate, ft³/day/lb substrate applied
G_{B} proportionality constant, lb/lb day
FSᵢ/XV=F/M=food to microorganism ratio, lb/lb day

Mathematical description of the specific methane production rate expressed as a function of the mass substrate loading rate for suspended growth systems follows:

$$M = \frac{{M}_{max} \times ( {FS}_{i} \text{/} XV )}{{M}_{B} + ( {FS}_{i} \text{/} XV )} \qquad ( 6 )$$

where
M=specific methane production rate, ft³/day/lb substrate applied
Mₘₐₓ=maximum specific methane production rate, ft/day/lb substrate applied
M_{B}=proportionality constant, lb/lb day

Equations 5 and 6 can be used to predict the total biogas and methane production at any given substrate loading rate. The biogas and methane biokinetic constants are determined in the same manner as the substrate biokinetic constants by plotting the reciprocals of the biogas and methane production rates as functions of the reciprocal of the substrate loading rate.

Mathematical description of the biogas and production rates can also be modeled as the substrate loading rate changes by using monomolecular kinetics in fixed-film anaerobic reactors. Total specific biogas production rate expressed as a function of the mass substrate loading rate follows for fixed-film anaerobic systems:

$$G = \frac{{G}_{max} ( {FS}_{i} \text{/} A )}{GB + ( {FS}_{i} \text{/} A )} \qquad ( 7 )$$

where
G=specific biogas production rate, ft³/day/1,000 ft²
Gₘₐₓ=maximum specific biogas production rate, ft³/day/1,000 ft²
G_{B}=proportionality constant, lbs substrate/day/1,000 ft²
FSᵢ/A=applied substrate loading rate, as previously described, lbs substrate/day/1,000 ft²
   The specific methane production rate expressed as a function of the mass substrate loading rate for fixed-film anaerobic systems follows:

$$M = \frac{{M}_{max} ( {FS}_{i} \text{/} A )}{{M}_{B} + ( {FS}_{i} \text{/} A )} \qquad ( 8 )$$

### Where

M=specific methane production rate, ft³/day/1,000 ft²
Mₘₐₓ=maximum specific methane production rate, ft³/day/1,000 ft²
M_{B}=proportionality constant, lbs substrate/day/1,000 ft²

Equations 7 and 8 can be used to predict the total biogas production and methane production in a fixed-film anaerobic system at any given substrate loading rate.

The biogas and methane kinetic constants are determined in the same manner as these kinetic constants for the anaerobic suspended growth system. Both the biogas and methane production data are plotted as a function of the substrate loading rate or mass COD loading rate as monomolecular kinetics. These kinetic plots are then linearized by plotting the reciprocal biogas and methane production rates as functions of the reciprocal COD mass loading rates.

Thus, input parameters in the process include substrate COD and/or VS (measures of substrate strength of biomethane generation potential). Additional input parameters include biokinetic constants:

Substrate treatment performance: Uₘₐₓ and K_{B}
Biogas production rate: Gₘₐₓ and G_{B}
Methane production rate: Mₘₐₓ and M_{B}

These constants mathematically model substrate utilization rate and conversion of substrate to biogas and methane. Since these biokinetic constants model substrate conversion to biogas and methane they can be used to predict biogas and methane production performance and when compared to actual measured values they determine the condition of the digestion system and whether the digester is performing as it should, or if operational changes are needed.

Additional input parameters include VFAs and alkalinity. These parameters (in conjunction with the on-line monitoring parameters) monitor the health of the digestion process and are used to determine if substrate feed rates should be decreased, remain the same, or can be increased.

The present invention provides an improved process and apparatus (digester reactor) for anaerobic biochemical digestion of waste, feedstocks, substrates (individually, or combined co-digestion) in various forms (e.g., soluble, slurry, particulate/solid or combination forms). The invention offers significant advantages, compared with prior art, in terms of digester reactor design, treatment performance, process optimization, maximum biogas production, and enhanced and optimized biomethane production.

It will be clear that the present invention is well adapted to attain the ends and advantages mentioned as well as those inherent therein. While presently preferred embodiments have been described for purposes of disclosure, numerous changes may be made which will readily suggest themselves to those skilled in the art and which are encompassed in the spirit of the invention disclosed and as defined in the appended claims.

What is claimed is:

1. An anaerobic process for producing biogas, comprising the steps of:
injecting feed material into a reactor having anaerobic microorganisms to form a volume of bulk liquid in the reactor;
monitoring the oxidation-reduction potential, the pH, and temperature of the reactor bulk liquid to determine whether oxidation-reduction potential, pH, and temperature are each within a predetermined range;
adjusting the amount of feed material fed to the reactor in response to a determination that one of the oxidation-reduction potential, pH, and temperature of the reactor bulk liquid are outside the corresponding predetermined ranges to adjust the oxidation-reduction potential, pH, or temperature of the reactor bulk liquid to within the predetermined range; and
producing biogas having methane.

2. The anaerobic process of claim 1, further comprising a step of:
maintaining the pH of the reactor bulk liquid within a range of from about 6.5 to about 8.5.

3. The anaerobic process of claim 1, further comprising a step of:
maintaining the oxidation-reduction potential of the reactor bulk liquid between about −300 mV and about −400 mV.

4. The anaerobic process of claim 1, wherein the process in the reactor is performed mesophilic at temperatures between about 80° F. and about 100° F.

5. The anaerobic process of claim 1, wherein the process in the reactor is performed thermophilic at temperatures between about 125° F. and about 150° F.

6. The anaerobic process of claim 1, further comprising the step of:
monitoring rate of the reactor biogas production to determine whether rate of production of the biogas is within a predetermined range.

7. The anaerobic process of claim 1, further comprising the step of:
monitoring relative rates of methane, carbon dioxide, and hydrogen sulfide production of the reactor biogas to determine whether methane, carbon dioxide, and hydrogen sulfide production are each within a predetermined range.

8. The anaerobic process of claim 7, wherein methane constitutes between about 60% and about 85% of the biogas.

9. The anaerobic process of claim 7, wherein the carbon dioxide constitutes between about 15% and about 40% of the biogas.

10. The anaerobic process of claim 1, further comprising the steps of:
removing treated effluent from the reactor; and
recycling at least a portion of the treated effluent to the reactor to increase biomass inventory within the reactor.

11. The anaerobic process of claim 10, further comprising the step of:
passing treated effluent through a solids/liquid separation apparatus for further treatment of the effluent.

12. An anaerobic process for producing biogas, comprising the steps of:
injecting an influent mixture into a reactor having anaerobic microorganisms to form a volume of bulk liquid in the reactor, wherein the influent mixture comprises a feed material and recycled effluent;
monitoring oxidation-reduction potential, pH, and temperature of the reactor bulk liquid;
adjusting at least a selected one of the rate of Injection of the recycled effluent and the amount of the feed material fed to the reactor in response to the oxidation-reduction potential, pH, and temperature of the reactor bulk liquid to maintain the temperature of the reactor bulk liquid within a predetermined range; and
producing biogas having methane.

13. The anaerobic process of claim 12, further comprising a step of:
maintaining the pH of the reactor bulk liquid within a range of from about 6.5 to about 8.5.

14. The anaerobic process of claim 12, further comprising a step of:
maintaining the oxidation-reduction potential of the reactor bulk liquid between about −300 mV and about −400 mV.

15. The anaerobic process of claim 12, wherein the process in the reactor is performed thermophilic at temperatures between about: 125° F. and about 150° F.

16. The anaerobic process of claim 12, wherein the process in the reactor is performed mesophilic at temperatures between about 80° F. and about 100° F.

17. The anaerobic process of claim 12, further comprising the step of:
monitoring rate of the reactor biogas production to determine whether rate of production of the biogas is within a predetermined range.

18. The anaerobic process of claim 12, further comprising the step of:
monitoring relative rates of methane, carbon dioxide, and hydrogen sulfide production of the reactor biogas to determine whether methane, carbon dioxide, and hydrogen sulfide production are each within a predetermined range.

19. The anaerobic process of claim 18, wherein methane constitutes between about 60% and about 85% of the biogas.

20. The anaerobic process of claim 18, wherein the carbon dioxide constitutes between about 15% and about 40% of the biogas.
